(19) United States
(12) Patent Application Publication
(10) Pub. No.: US 2020/0375190 A1
(43) Pub. Date: Dec. 3, 2020

(54) Alkyl Dimethyl Organosilane Quaternaries in Persistent Systems and Methods

(71) Applicant: Nano Global, Austin, TX (US)

(72) Inventors: Christine Scheve, Austin, TX (US); William Peterson, III, Austin, TX (US); Zoltan Papp, Austin, TX (US); Stephen Smid, Austin, TX (US)

(21) Appl. No.: 16/636,137

(22) PCT Filed: Jul. 31, 2018
(86) PCT No.: PCT/US2018/044651

§ 371 Date: Feb. 3, 2020

Related U.S. Application Data
(60) Provisional application No. 62/540,038, filed on Aug. 1, 2017.

(51) Int. Cl. A01N 55/00; A61K 31/695; A61K 9/08; A61K 9/06; A61K 8/58; A61Q 11/00; A61L 31/08; A61L 29/08; A61L 27/28; A61Q 15/00

(52) U.S. Cl. CPC A01N 55/00; A61K 31/695; A61K 9/08; A61K 9/06; A61Q 15/00; A61Q 11/00; A61L 31/08; A61L 29/08; A61L 27/28; A61K 8/585

(57) ABSTRACT

The technology described herein includes methods and uses for quaternary ammonium, specifically an organosilane quaternary ammonium or quat, and particularly AIVIOSILQ™ quat to provide persistent disinfectant and anti-microbial protection. Uses include skin (derma) and medical applications.

### RELATED APPLICATIONS

This application claims the benefit of priority of U.S. Provisional Patent Application Serial No. 62/540,038 filed Aug. 1, 2017, incorporated herein by reference.

### BACKGROUND

Benzyl ammonium quaternary chlorides (BAC) are effective antimicrobial compounds. Current BAC formulations been found to be most effective as antimicrobial agents as mixtures of various alkyl dimethyl chain lengths. Other well-known quaternary ammonium compounds or “quats” are known to have disinfectant properties and are used in various settings. Quat-based disinfectants and sanitizers may be used on skin and hard non-porous surfaces to kill and prevent the spread of infectious agents including bacteria, viruses, fungus, algae, and the like. Quat-based disinfectants may be used in healthcare, hospitality, schools, agriculture, as well as various other locations and settings where killing and reducing the spread of infectious agents is desired.

Many skin and surface disinfectants are capable of disinfecting or treating immediately; however, the effectiveness is short lived and at times immediately goes away after application. Therefore, skin and surface treatments, along with disinfected surfaces that keep a persistent or long-term state of effectiveness are desirable.

### DETAILED DESCRIPTION

Described herein are uses, systems and methods for the use of a quatemary ammonium or “quat”, and in particular, octadecyldimethyl (3-trihydroxypropyl) ammonium chloride, or the trademarked AMOSILQTM “quat”.

The AMOSILQ™ “quat” may be applied or be used in various forms such as the following described implementations. For example:

### USE FOR SKIN AND SURFACES

### ALCOHOL AND ORGANOSILANE COMBO ON SKIN:

Increased binding, homogeneity (minimal to no domain formation once dry) and antimicrobial efficacy of self-assembled organosilane quaternary based polymeric barrier on skin due to the presence of varying percentages of ethanol and isopropyl alcohol. Example formulation ranges includes up to 70% alcohol in solution, and organosilane quaternary of up to 10% in solution, based on solubility in given formulation and amount required for desired antimicrobial efficacy. Formulation is also based on ranges within both hand and surface applications.
Better binding and be more effective as an antimicrobial in the presence of ethanol and or isopropyl alcohol. This combined effect could result in higher/broader spectrum of kill rates both in solution and once the organosilane quatemary binds to the surface. Once dried, the organosilane quaternary compound forms an organized antimicrobial layer capable of weakening/destroying the cell membrane of the target organism. Alcohol works to denature all proteins once the membrane has been compromised. Together, the overall antimicrobial efficacy can be enhanced. Alcohol also increases the stability and drying time of the self-assembled polymeric nano-barriers formed, thus enhancing binding, antimicrobial efficacy and decreasing domain formation on the surface.
These functions can also be applied to non-living porous and non-porous substrates
Surfaces can include metal, glass, plastics, rubber, ceramics and fabrics including cellulose, cotton, acetates, nylon, etc.

### Applications:

DUAL FUNCTIONING HAND WASH AND RUB PRODUCT:
   Formulation can act as both an antiseptic hand wash and rub (hand sanitizer without water). This can be a two-step process from the same bottle. Step one) Wash hands with product to remove unwanted grim. Step 2) Pump and rub on hands for long lasting antimicrobial protection
OVER THE COUNTER SKIN APPLICATIONS
   TOPICAL ANTIMICROBIAL: HEALTHCARE, FIRST AID AND CONSUMER ANTISEPTICS
      Purposes:
         Consumer hand rub
         Consumer hand wash
         First aid hand rub
         First aid hand wash
         Patient preoperative skin preparation
         Healthcare professional hand rub
         Healthcare professional hand wash
         Surgical hand scrub
         Surgical hand rub
      Potential forms:
         Liquid
         Gel
         Spray
         Foam
      Potential active ingredients to be combined with organosilane quaternary ammonium compound such as (3-(Trimethoxysil)Propyl)OctadecyDimethylAmmonium Chloride:
         Alcohol, ethanol, ethyl alcohol
         Benzalkonium chloride
         Benzethonium chloride
         Chlorhexidine gluconate
         Chloroxylenol
         Iodine tincture USP
         Iodine topical solution USP
         Isopropyl alcohol
         Mercufenol chloride
         Combinations:
         Calomel, oxyquinoline benzoate, triethanolamine, and phenol derivative
         Mercufenol chloride and secondary amyltricresols in 50 percent alcohol
   TOPICAL SKIN PROTECTANT AND ANTIMICROBIAL FOR DIAPER RASH
      Purpose:
         Helps treat and prevent diaper rash
         Antimicrobial
         Helps protect minor skin irritation due to diaper rash and helps seal out wetness
      Potential forms:
         Spray
         Cream
         Ointment
         Powder
      Potential active ingredients to be combined with organosilane quaternary ammonium compound such as (3-(Trimethoxysil)Propyl)OctadecyDimethylAmmonium Chloride:
         Alkyldimethyl Benzylammonium chloride
         Allantoin (5-ureidohydantoin)
         Aluminum acetate
         Aluminum hydroxide
         Amylum
         Balsam peru
         Benzethonium chloride
         Benzocaine
         Bicarbonate of soda
         Bismuth subnitrate
         Boric acid
         Calamine
         Calcium carbonate
         Camphor
         Casein
         Cod liver oil
         Cysteine hydrochloride
         Dibucaine
         Diperodon hydrochlorid
         Glycerin
         Hexachlorophene
         8-hydroxyquinoline
         Iron oxide
         Lanolin
         Menthol
         Methapyrilene
         Methionine
         Methylbenzethonium chloride
         Oil of eucalyptus
         Oil of lavender
         Oil of peppermint
         Oil of white thyme
         Panthenol
         Para-chloromercuriphenol
         Petrolatum
         Phenol
         Pramoxine hydrochloride
         Salicylic acid
         Silicone
         Sorbitan monosterate
         Talc
         Tetracaine
         Vitamin A
         Vitamin A palmitate
         Vitamin D
         Vitamin D2
         Vitamin E
         White petrolatum
         Zinc oxide
         Zinc stearate
   TOPICAL FIRST AID ANTIBIOTIC DRUG PRODUCTS
      Purpose:
         Help prevent infection in minor cuts, scrapes, and bums
      Potential forms:
         Cream
         Ointment
      Potential active ingredients to be combined with organosilane quaternary ammonium compound such as (3-(Trimethoxysil)Propyl)OctadecyDimethylAmmonium Chloride:
         Bacitracin ointment
         Bacitracin zinc
         Chlortetracycline hydrochloride
         Neomycin sulfate
         Tetracycline hydrochloride
         Oxytetraclyine hydrochloride
   TOPICAL ANTIFUNGAL DRUG PRODUCTS
      Purpose:
         Inhibits the growth and reproduction of fungal cells and deceases the number of fungi present
         To treat athletes foot, jock itch ringworm
      Potential forms:
         Cream
         Gel
         Aerosol
      Potential active ingredients that can be combined with organosilane quaternary ammonium compound such as (3-(Trimethoxysil)Propyl)OctadecyDimethylAmmonium Chloride:
         Clioquinol
         Haloprogin
         Miconazole nitrate
         Povidone-iodine
         Tolnaftate
         Undecylenic acid
         Calcium undecylenate
         Copper undecylenate
         Zinc undecylenate
   TOPICAL ACNE DUG PRODUCTS
      Purpose:
         Acne medication, acne treatment
         Formulation form:
         Lotion
         Cream
         Ointment
         Gel
      Potential active ingredients to be combined with organosilane quaternary ammonium compound such as (3(Trimethoxysil)Propyl)OctadecyDimethylAmmonium Chloride:
         Resorcinol
         Resorcinol monoacetate
         Salicylic acid
         Sulfur
   TOPICAL SKIN PROTECTANT: DEODORANT
      Purpose:
         antiseptic, antiperspirant
      Formulation form:
         Solid
         Gel
         Liquid
      Potential active ingredients to be combined with organosilane quaternary ammonium compound such as (3-(Trimethoxysil)Propyl)OctadecyDimethylAmmonium Chloride:
         Sodium stearate
         Sodium chloride
         Stearyl alcohol
         Potassium alum
         Aluminum Zirconium
         Aluminum Chlorohydrate
         Aluminum chloride
   TOPICAL: DENTAL
      Purpose:
         Antiseptic, Anti-gingivitis, anti-plaque, anti-cavity
      Formulation form:
         Liquid
         Spray
         Paste
         Gel
      Potential active ingredients to be combined with organosilane quaternary ammonium compound such as (3-(Trimethoxysil)Propyl)OctadecyDimethylAmmonium Chloride:
         Sodium fluoride
         Sodium monofluorophoshate
         Hydrogen peroxide
         Stannoud fluoride
         Cetylpyridinium chloride
         Thymol
         Menthol
         Eucalyptol
         Methyl salicylate

### MEDICAL DEVICE COATINGS

The application of organosilane compound alone or in combination with other coatings for enhanced, long lasting, non-leaching antimicrobial properties polymeric nano-barriers formed, thus enhancing binding, antimicrobial efficacy and decreasing domain formation on the surface.
Length of antimicrobial protection depends on application, exposure to living system parameters (heat, chemistry, flow), surface cleaning agents and overall exposure to abrasion
Organosilane can be embedded during manufacturing of devices, coated post production or applied in clinical setting
Additional favorable biological interfacing properties also possible including reduced inflammation and immune response after implantation

### DISPOSABLE DEVICE PACKAGING

### NON-SURGICAL/NON-IMPLANT ABLE DEVICES

Personal Protective Equipment
   Gloves
   Personnel hair, nose and mouth protectors
   Scrubs and gowns
   Goggles
Wound care
   Bandages/ wraps
Orthopedic
   Braces/casting
Exam room equipment
Laboratory equipment
Diagnostic modality components
EMS/patient transport equipment

### SURGICAL EQUIPMENT

Surgical tools
Surgical tool packaging
Surgical furniture

### IMPLANTABLE DEVICES

Stents
Pacemakers
Bladder catheters
Central venous catheters
Fracture fixation devices
Dental implants
Joint prostheses
Vascular grafts
Cardiac pacemakers
Mammary implants
Mechanical heart valves

What is claimed is:

1. A method of using an organosilane quaternary ammonium comprising:
combining the organosilane quaternary ammonium with ethanol and/or isopropyl alcohol into a solution; and
applying the combined solution on skin and/or surfaces.

2. A method of using a quaternary ammonium solution as a hand wash and disinfectant rub comprising:
combining a quaternary ammonium in a solution to be used as the hand wash and disinfectant rub.

3. A method of using an organosilane quaternary ammonium in a topical application comprising:
using the organosilane quaternary ammonium in one or more of the following:
Consumer hand rub
Consumer hand wash
First aid hand rub
First aid hand wash
Patient preoperative skin preparation
Healthcare professional hand rub
Healthcare professional hand wash
Surgical hand scrub
Surgical hand rub

4. The method of using an organosilane quaternary ammonium in a topical application of claim 3, wherein the topical application is in the form of one of the following:
Liquid
Gel
Spray
Foam

5. The method of using an organosilane quaternary ammonium in a topical application of claim 3, wherein the topical application is combined with one of the following:
a. Alcohol, ethanol, ethyl alcohol
b. Benzalkonium chloride
c. Benzethonium chloride
d. Chlorhexidine gluconate
e . Chloroxylenol
f. Iodine tincture USP
g. Iodine topical solution USP
h . Isopropyl alcohol
i. Mercufenol chloride
j . Combinations:
i. Calomel, oxyquinoline benzoate, triethanolamine, and phenol derivative
ii. Mercufenol chloride and secondary amyltricresols in 50 percent alcohol

6. A method for using an organosilane quaternary ammonium comprising:
applying the organosilane quaternary ammonium in a topical solution to prevent diaper rash and seal out moisture

7. The method of claim 6, wherein the topical solution is in the form of a spray, cream, ointment, or powder.

8. The method of claim 6, wherein the topical solution is combined with one of the following:
Alkyldimethyl Benzylammonium chloride
Allantoin (5-ureidohydantoin)
Aluminum acetate
Aluminum hydroxide
Amylum
Balsam peru
Benzethonium chloride
Benzocaine
Bicarbonate of soda
Bismuth subnitrate
Boric acid
Calamine
Calcium carbonate
Camphor
Casein
Cod liver oil
Cysteine hydrochloride
Dibucaine
Diperodon hydrochlorid
Glycerin
Hexachlorophene
8-hydroxyquinoline
Iron oxide
Lanolin
Menthol
Methapyrilene
Methionine
Methylbenzethonium chloride
Oil of eucalyptus
Oil of lavender
Oil of peppermint
Oil of white thyme
Panthenol
Para-chloromercuriphenol
Petrolatum
Phenol
Pramoxine hydrochloride
Salicylic acid
Silicone
Sorbitan monosterate
Talc
Tetracaine
Vitamin A
Vitamin A palmitate
Vitamin D
Vitamin D2
Vitamin E
White petrolatum
Zinc oxide
Zinc stearate

9. A method for using an organosilane quaternary ammonium comprising:
applying the organosilane quaternary ammonium as a topical first aid antibiotic.

10. The method of claim 9, wherein the topical first aid antibiotic is in the form of a cream or ointment.

11. The method of claim 9, wherein the topical first aid antibiotic includes one of the following:
Bacitracin ointment
Bacitracin zinc
Chlortetracycline hydrochloride
Neomycin sulfate
Tetracycline hydrochloride
Oxytetraclyine hydrochloride

12. A method for using an organosilane quaternary ammonium comprising:
applying the organosilane quaternary ammonium as an antifungal.

13. The method of claim 12, wherein the topical first aid antibiotic is in the form of a cream, gel or aerosol.

14. The method of claim 12, wherein the topical first aid antibiotic includes one of the following:
Clioquinol
Haloprogin
Miconazole nitrate
Povidone-iodine
Tolnaftate
Undecylenic acid
Calcium undecylenate
Copper undecylenate
Zinc undecylenate

15. A method for using an organosilane quaternary ammonium comprising:
applying the organosilane quaternary ammonium as a treatment for acne.

16. The method of claim 14, wherein the topical first aid antibiotic is in the form of a lotion, cream, ointment or gel.

17. The method of claim 14, wherein the topical first aid antibiotic includes one of the following:
Resorcinol
Resorcinol monoacetate
Salicylic acid
Sulfur

18. A method for using an organosilane quaternary ammonium comprising:
applying the organosilane quaternary ammonium as a skin deodorant.

19. The method of claim 18, wherein the topical first aid antibiotic is in the form of a solid, gel, or liquid.

20. The method of claim 18, wherein the topical first aid antibiotic includes one of the following:
Sodium stearate
Sodium chloride
Stearyl alcohol
Potassium alum
Aluminum Zirconium
Aluminum Chlorohydrate
Aluminum chloride

21. A method for using an organosilane quaternary ammonium comprising:
applying the organosilane quaternary ammonium as a dental product.

22. The method of claim 21, wherein the topical first aid antibiotic is in the form of a liquid, spray, paste or gel

23. The method of claim 21, wherein the topical first aid antibiotic includes one of the following:
Sodium fluoride
Sodium monofluorophoshate
Hydrogen peroxide
Stannoud fluoride
Cetylpyridinium chloride
Thymol
Menthol
Eucalyptol
Methyl salicylate

24. A method for using an organosilane quaternary ammonium comprising:
applying to medical device coatings.

25. A method for using an organosilane quaternary ammonium comprising:
applying to disposable device packaging.

26. A method for using an organosilane quaternary ammonium comprising:
applying to non surgical/non implantable devices.

27. The method of claim 26, wherein the non-surgical/non-implantable devices include one or more of the following:
Personal Protective Equipment
Gloves
Personnel hair, nose and mouth protectors
Scrubs and gowns
Goggles
Wound care
Bandages/ wraps
Orthopedic
Braces/casting
Exam room equipment
Laboratory equipment
Diagnostic modality components
EMS/patient transport equipment

28. A method for using an organosilane quaternary ammonium comprising:
applying to surgical equipment.

29. The method of claim 28, wherein the surgical equipment includes one or more of the following:
Surgical tools
Surgical tool packaging
Surgical furniture

30. A method for using an organosilane quaternary ammonium comprising:
applying to implantable devices.

31. The method of claim 30, wherein the surgical equipment includes one or more of the following:
Stents
Pacemakers
Bladder catheters
Central venous catheters
Fracture fixation devices
Dental implants
Joint prostheses
Vascular grafts
Cardiac pacemakers
Mammary implants
Mechanical heart valves
